(19) United States
(12) Patent Application Publication
(10) Pub. No.: US 2010/0269825 A1
(43) Pub. Date: Oct. 28, 2010

(54) INHALATION PARTICLES COMPRISING A SALT OF 8-HYDROXY-2-[[(1R)-2-(4-METHOXYPHENYL)-1-METHYLETHYL]AMINO] ETHYL]-2(1H)-QUINOLINONE AND A CORTICOSTEROID

(75) Inventors: Daniela COCCONI, Parma (IT); Rossella MUSA, Parma (IT); Gaetano BRAMBILLA, Parma (IT); Dipesh PARIKH, Oxford (GB); Graham RUECROFT, Oxford (GB)

Correspondence Address: OBLON, SPIVAK, MCCLELLAND MAIER & NEUSTADT, L.L.P., 1940 DUKE STREET, ALEXANDRIA, VA, 22314, US

(73) Assignee: Chiesi Farmaceutici S.p.A., Parma (IT)

(21) Appl. No.: 12/712,531

(22) Filed: Feb. 25, 2010

(30) Foreign Application Priority Data
Feb. 25, 2009 (EP) 09153648.2

(51) Int. Cl. A61M 15/00; A61K 31/573; A61K 9/12; A61P 11/06

(52) U.S. Cl. 128/203.15; 514/171; 424/43

(57) ABSTRACT

Crystalline particles wherein each particle comprises a combination of a pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone (carmoterol), and a corticosteroid in a pre-determined and constant ratio are effective for the prevention and treatment of inflammatory or obstructive airways diseases.

### CROSS REFERENCES TO RELATED APPLICATIONS

This application claims priority to European Patent Application No. 09153648.2, filed on Feb. 25, 2009, which is incorporated herein by reference in its entirety.

### BACKGROUND OF THE INVENTION

1. Field of the Invention

The present invention relates to crystalline particles wherein each particle comprises a combination of a pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone (carmoterol), and a corticosteroid in a pre-determined and constant ratio. The present invention also relates to a method for preparing such particles and to inhalation compositions which contain such particles. The present invention also relates to the treatment of respiratory diseases such as asthma and COPD by the administration of such particles.

2. Discussion of the Background

The administration of pharmacologically active ingredients by inhalation to the airways is a widely used technique especially for the prevention and/or treatment of broncho-pulmonary diseases. The most widely used systems for the administration of drugs to the airways are the dry powder inhalers (DPIs), which comprise micronized drug particles as a dry powder usually admixed with coarser excipient particles of pharmacologically inert materials such as lactose, and the pressurized metered-dose inhalers (pMDIs) which may comprise a suspension of micronized drug particles in a propellant gas.

Drugs commonly delivered by inhalation for treating broncho-pulmonary diseases such as asthma and chronic obstructive pulmonary disease (COPD) include long-acting beta₂-agonists (LABA) such as formoterol and salmeterol, and inhaled corticosteroids (ICS) such as beclometasone dipropionate (BDP), budesonide, mometasone furoate, ciclesonide, and fluticasone propionate. To improve the compliance of the patients, formulations have been developed which comprise a combination of LABA and ICS, thereby reducing the number of inhalers that the patients would normally require.

On the other hand, said formulations usually combine the LABA and ICS only as far as creating a physical mixture of the two separate drugs with or without excipients. When these combinations are used in said kind of formulations, the method of mechanically mixing the two different drugs has certain drawbacks. For example, the consistency of drug proportion in each dose cannot be easily controlled. The ratio of drugs in each dose significantly depends on the forces existing between the drugs, and between each drug and the excipients. In this respect, it is well known that the manufacturing methods currently used, such as conventional milling processes, produce very cohesive particles.

Another factor that jeopardizes the possibility of maintaining the ratio of the drugs in each dose constant is the size of the drug particles. Said parameter indeed cannot easily be controlled, and hence it is difficult obtain drugs having the same particle size.

The inconsistency of the dose could cause serious problems as it could give rise to a risk of an over or under dosage. In particular, ensuring a good constancy of the ratio of the drugs ratio is very critical for combinations with very low-dosage strength LABA drugs which are present in the formulation in a very low concentration. In fact, the lesser is the LABA concentration, the greater the ratio between said drug and the ICS in the formulation, and hence more difficult is maintaining the constancy of the ratio.

For example 8-hydroxy-5-[(1R)-1-hydroxy-2-[[(1R)-2-(4-methoxyphenyl)-1-methylethyl]amino]ethyl]-2(1H)-quinolinone (hereinafter indicated as carmoterol), is a very low-dosage strength LABA which is to be administered by inhalation at a very low daily therapeutical dose ranging from 1 to 4 μg. It is currently under development in the form of a pharmaceutically acceptable salt in combination with budeosonide in ratios in which the corticosteroid is the overwhelming part. These facts, together with other properties such as his high adhesiveness degree, lead to problems in the manufacturing of compositions comprising carmoterol in combination with a ICS wherein the two drugs are present in a constant ratio in each delivered dose.

Moreover it would be highly preferable to provide formulations wherein both active ingredients are in crystalline form, especially with regard to suspension based-pMDI and DPI formulations. As matter of fact, the presence of amorphous material may lead to batch-to-batch variation in the performance during product's lifespan in addition to physical and chemical stability problems.

### SUMMARY OF THE INVENTION

Accordingly, it is one object of the present invention to provide novel crystalline particles which comprise a combination of a pharmaceutically acceptable salt of carmoterol, and a corticosteroid in a predetermined and constant ratio.

It is another object of the present invention to provide novel methods for preparing such particles.

It is another object of the present invention to provide novel methods for treating and/or preventing broncho-pulmonary diseases by administering such particles.

These and other objects, which will become apparent during the following detailed description, have been achieved by the inventors' discovery of crystalline particles for use in pharmaceutical formulations for inhalation, wherein each particle comprises a combination of a pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone (carmoterol), and a corticosteroid in a ratio of no more than 1:50.

In another aspect the present invention provides a process for preparing such crystalline particles wherein each particle comprises a combination of a pharmaceutically acceptable salt of carmoterol, and a corticosteroid in a ratio of no more than 1:50, said process comprising the steps of:

a) forming of a solution of the two different active ingredients in a pre-determined ratio in a suitable solvent;

b) generating an aerosol from the solution of said two active ingredients;

c) collecting of the aerosol droplets in a vessel containing an anti-solvent for both the active ingredients;

d) applying high intensity ultrasound to induce crystallisation of said two active ingredients into an individual particle; and

e) isolating and collecting the produced particles.

Preferably the solvent is a mixture of dichloromethane and methanol in a ratio comprised between 90:10 and 99:1 v/v, and the anti-solvent is n-heptane.

In a further aspect, the present invention provides a process for preparing such crystalline particles wherein each particle comprises a combination of a pharmaceutically acceptable salt of carmoterol, and a corticosteroid in a ratio of no more than 1:50, said process comprising the steps of:

a) forming of a solution of the two active ingredients in a pre-determined ratio in a suitable solvent;

b) generating an aerosol from the solution of said two active ingredients and drying the atomized droplets to yield unstable part-amorphous or fully amorphous particles;

c) collecting of the particles in a vessel containing an anti-solvent for both the active ingredients;

d) applying high intensity ultrasound to induce crystallisation of said two active ingredients into an individual particle; and

e) isolating and collecting the produced particles.

Preferably the solvent is methanol.

The present invention also provides crystalline particles for use in pharmaceutical formulations for inhalation, wherein each particle comprises a combination of a pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone (carmoterol), and a corticosteroid in a ratio of no more than 1:50, said particles obtainable by a process comprising:

a) forming of a solution of the two different active ingredients in a pre-determined ratio in a suitable solvent;

b) generating an aerosol from the solution of said two active ingredients;

c) collecting of the aerosol droplets in a vessel containing an anti-solvent for both the active ingredients;

d) applying high intensity ultrasound to induce crystallisation of said two active ingredients into an individual particle; and

e) isolating and collecting the produced particles.

Alternatively, said particles are obtainable by a process comprising:

a) forming of a solution of the two active ingredients in a pre-determined ratio in a suitable solvent;

b) generating an aerosol from the solution of said two active ingredients and drying the atomized droplets to yield unstable part-amorphous or fully amorphous particles;

c) collecting of the particles in a vessel containing an anti-solvent for both the active ingredients;

d) applying high intensity ultrasound to induce crystallisation of said two active ingredients into an individual particle; and

e) isolating and collecting the produced particles.

In another aspect, the present invention provides a formulation for administration by inhalation comprising the aforementioned particles, optionally together with one or more pharmaceutically acceptable excipients.

Preferably, the formulation is provided in the form of dry inhalation powder to be used with dry powder inhaler (DPI) devices or in the form of a suspension of the particles in a propellant gas to be used with pressurized metered-dose inhaler (pMDI) devices.

Therefore the present invention also provides a device which may be a single- or multi-dose dry powder inhaler, or a pressurized metered dose inhaler, respectively, filled with the aforementioned formulations.

In another aspect, the present invention provides the use of the crystalline particles of the invention as a medicament.

In a further aspect the invention provides the use of the aforementioned particles for the prevention and/or treatment of an inflammatory or obstructive airways disease such as asthma or chronic obstructive pulmonary disease (COPD).

In a still further aspect, the present invention provides a method of preventing and/or treating an inflammatory or obstructive airways disease such as asthma or chronic obstructive pulmonary disease (COPD), which comprises administration by inhalation of an effective amount of the crystalline particles of the invention.

Said particles allow preparing formulations for inhalation with improved properties in terms dosing compliance. Since said particles make it possible to achieve a co-deposition at the target cell of the lungs of the combination of the active drug substances, the relevant formulations may also give rise to superior therapeutic benefit due to the enhanced synergistic action.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a SEM image of particles consisting of carmoterol hydrochloride and budesonide in a ratio 1:100 w/w.

FIG. 2 shows SEM images of carmoterol hydrochloride (left) and budesonide (right) raw materials.

FIG. 3 is a X-ray powder diffraction pattern of particles consisting of carmoterol hydrochloride and budesonide in a ratio 1:100 w/w.

FIG. 4 is a thermogram of particles consisting of carmoterol hydrochloride and budesonide in a ratio 1:100 w/w (solid line) in comparison to pure crystalline budesonide (dash line) and pure crystalline carmoterol hydrochloride (dash-dot line).

### DETAILED DESCRIPTION OF THE PREFERRED EMBODIMENTS

In the context of the present invention, the terms “drug,” “active ingredient,” “active agent.” and ‘“active substance” are used as synonyms.

In the context of the present invention, the term “solvent” is used to mean the medium in which the active ingredient is dissolved and “anti-solvent” is used to mean the medium in which its crystallization takes place.

The term “very low-dosage strength” refers to active ingredients endowed with particularly high potency which are present in the powder formulation in a very low concentration. Such active ingredients are commonly administered at a daily therapeutic dose lower than 6 μg.

By “daily therapeutically effective dose,” it is meant the quantity of active ingredient administered in one day by inhalation. Said daily dose may be delivered in one or more administrations per day and in one or more actuations of the inhaler per administration.

By “actuation,” it is meant the release of active ingredient from the device by a single activation (e.g. mechanical or breath).

The daily dose may be reached by a single or double administration.

In one embodiment, the daily dose may be reached by a single administration and delivered in one actuation of the inhaler.

In another preferred embodiment, the daily dose may be reached by a single administration and delivered in more actuations of the inhaler, preferably two.

In a further embodiment, the daily dose may be reached by a double administration and each delivered in one actuation of the inhaler.

In an even further embodiment, the daily dose may be reached by a double administration and delivered in more actuations of the inhaler, preferably two.

In the context of the present invention the expression “each particle comprises a combination of a pharmaceutically acceptable salt of carmoterol and a corticosteroid” means that a single unagglomerated particle, whose size is in the range of microns, comprises a crystalline corticosteroid in which carmoterol, being present in a low amount, is incorporated as “an impurity”. This phenomenon, known as “crystal doping” is demonstrated by the depression of the melting point of the crystalline corticosteroid.

As used herein, the expression “good constancy of the drug ratio” means that the two active ingredients, after delivery of a single therapeutical dose, maintain substantially the same ratio as the pre-determined ratio of said two active ingredient in the formulation, i.e. that the relative standard deviation (RSD) of the ratio of the amounts of drugs measured in an in vitro apparatus such as the Andersen Cascade Impactor (ACI) is less is less than 15%, preferably less than 10%.

In general terms, the particle size is quantified by measuring a characteristic equivalent sphere diameter, known as volume diameter, by laser diffraction. The particle size may also be quantified by measuring the mass diameter by means of suitable instrument well known to the skilled person such as, for instance the sieve analyser. The volume diameter (VD) is related to the mass diameter (MD) by the density of the particles (assuming a size independent density for the particles).

In the present application, the particle size is expressed in terms of mass diameter and the particle size distribution is expressed in terms of: i) the volume median diameter (MVD) which corresponds to the diameter of 50 percent by weight or volume respectively, of the particles [d(v,0.5)], and ii) the MD in micron of 10% and 90% of the particles, respectively [d(v,0.1) and [d(v,0.9)].

Upon aerosolization, the particle size is expressed as mass aerodynamic diameter (MAD) which indicates the capability of the particles of being transported suspended in an air stream. The term MMAD stands for median mass aerodynamic diameter.

As used herein, the expression “good homogeneity” refers to a formulation wherein, upon mixing, the content uniformity of the active ingredient, expressed as relative standard deviation (RSD), is less than 5%, preferably less than 3%.

As used herein, the expression “respirable fraction” refers to an index of the percentage of active particles which would reach the deep lungs in a patient. The respirable fraction, also termed fine particle fraction, is evaluated using a suitable in vitro apparata such as the Andersen Cascade Impactor (ACI) or the Mutli Stage Liquid Impinger (MLSI) according to procedures reported in common Pharmacopoeias. It is calculated by the ratio between the respirable dose and the delivered dose. The delivered dose is calculated from the cumulative deposition in the apparatus, while the respirable dose (fine particle dose) is calculated from the deposition on Stages 3 (S3) to filter (AF) corresponding to particles≦5.0 microns. A respirable fraction higher than 30% is an index of good inhalatory performances.

As used herein the term “synergistic” means that the activity of the two active ingredients is more than would be expected by summing their respective individual activities in a given assay.

As used herein the term “interactive or ordered mixture” refers to powder formulation for inhalation comprising a pharmacologically-inert physiologically acceptable carrier substance, to which the micronized active compound particles are bonded by adhesion in order thus to achieve and to maintain a suitable mixed material, i.e. homogeneity of the mixture.

As used herein, the term “relatively highly fissured surface” means a surface on which there are clefts and valleys and other recessed regions, referred to herein collectively as fissures. Said surface of the coarse excipient particles may be defined in terms of fissure index or rugosity coefficients as disclosed in WO 01/78695 and WO 01/78693, which are incorporated herein by reference in their entireties, and they can be characterized according to the description therein reported.

The present invention provides crystalline particles for use in pharmaceutical formulations for inhalation, each of said particles comprising a combination of a pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone (carmoterol), and a corticosteroid in a constant ratio.

Upon inhalation, the particles of the invention provide more controlled delivery of the combination of carmoterol with a corticosteroid, since they allow keeping the ratio of the drugs constant upon each actuation.

The particles of the present invention are in a substantially crystalline form and show a reduce tendency of moisture adsorption as demonstrated in Example 3, contributing to increase their physical and chemical stability. Said particles also exhibit excellent dispersion properties making it easy to obtain homogenous formulations, in particular when the particles are formulated as dry powders for inhalation.

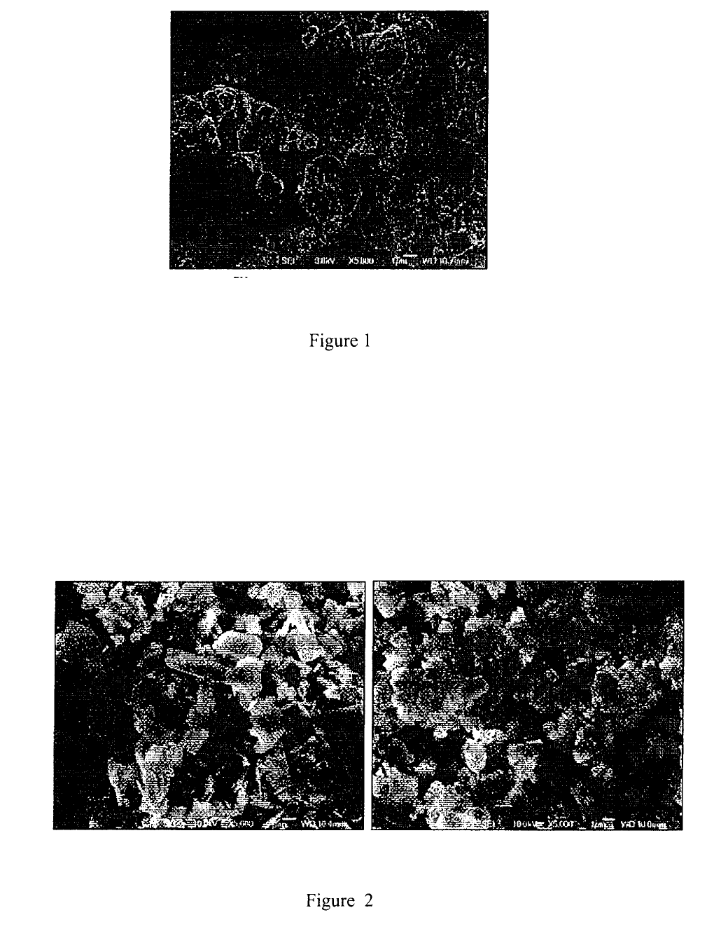

By scanning electron microscopy (SEM), it can be clearly observed that said particles are significantly distinct when compared to the SEM image of the starting materials (compare, FIGS. 1 and 2). It can also be appreciated that the particles of the present invention exhibit a more uniform and regular spherical shape and do not appear to be as fractured and irregular as the starting materials. The difference in the surface morphology contributes to lower the tendency of aggregation of the particles, and hence explain their excellent dispersion properties.

Examples of pharmaceutically acceptable salts of carmoterol include the hydrochloride, hydrobromide, sulfate, phosphate, maleate, fumarate, tartrate, citrate, benzoate, mesylate, ascorbate, salicylate, acetate, succinate, lactate, glutarate or gluconate. Preferably carmoterol is used in the form of hydrochloride salt.

Advantageously, the corticosteroid is any corticosteroid which is insoluble or poorly-soluble in water according to the definition of solubility given in the European Pharmacopoeia Ed. 4^{th}, 2002, and which can be utilized by inhalation for the prevention and/or treatment of respiratory diseases, and having a single therapeutic dose higher than 50 micrograms, preferably equal to or higher than 80 micrograms, more preferably equal to higher than 100 micrograms. Preferably, the corticosteroid is selected from the group consisting of beclometasone dipropionate (BDP), budesonide, ciclesonide, mometasone and esters thereof, e.g. furoate, fluticasone and esters thereof, e.g. propionate and furoate. In a preferred embodiment, of the invention the corticosteroid is budesonide.

The predetermined and constant ratio of carmoterol to the corticosteroid in the particles of the invention is no more than 1:50 expressed as w/w. Depending on the choice of the corticosteroid, it is advantageously from 1:50 to 1:800, preferably from 1:80 to 1:400.

For example, when budesonide is used, the w/w ratio may be from 1:50 to 1:400, preferably from 1:180 and 1:320. In one of the preferred embodiments of the invention, the w/w ratio is preferably about 1:100, while in another preferred embodiment the w/w ratio is about 1:160.

Another corticosteroid that can be advantageously used in the combination is mometasone furoate, and in this case the w/w ratio will range from 1:100 to 1:400.

The particles of the particles invention should have a narrow particle size distribution in a range suitable for their administration by inhalation.

Advantageously, the particles of the present invention have a particle size distribution lower than 15 microns, and more advantageously at least 90% of the particles have a diameter equal to or lower than 12 microns as determined by measuring the characteristic equivalent sphere diameter, known as volume diameter, by laser diffraction as described above, preferably using a Malvern or an equivalent apparatus.

Preferably, no more than 10% of said particles have a volume diameter [d(v,0.1)] lower than 0.8 microns, no more than 50% of have a volume diameter [d(v,0.5)] lower than 2.0 microns, and at least 90% have a volume diameter equal to or lower than 11 microns.

The particles of the present invention are substantially in a crystalline form. Advantageously the degree of crystallinity, expressed as weight % of the crystalline particle with respect to the total weight of the particle, is higher than 90%, preferably higher than 93%, even more preferably equal to or higher than 95%. The degree of crystallinity of the particle may be determined using X-ray powder diffraction or other techniques known to the skilled person such as microcalorimetry.

The active ingredients in the particles of the present invention are substantially in a pure form, e.g. both at least of 95% w/w, preferably 98% or 99% w/w or greater. The chemical purity may be determined according to methods known to the skilled person such as high-performance liquid chromatography (HPLC).

The particles of the present invention may be prepared using certain apparatus and techniques disclosed in the co-pending applications, WO 2004/073827 and WO 2010/00447, which are incorporated herein by reference in their entireties. Thus, in an aspect, and following the teaching of WO 2004/073827, the present invention provides a process for the production of the particles of the invention comprising:

a) forming of a solution of the two active ingredients (the salt of carmoterol and the corticosteroid) in a pre-determined ratio in a suitable solvent;

b) generating an aerosol from the solution of said two active ingredients;

c) collecting of the aerosol droplets in a vessel containing an anti-solvent for both the active ingredients;

d) applying high intensity ultrasound to induce crystallisation of said two active ingredients into an individual particle; and

e) isolating and collecting the produced particles.

In another aspect, and following the teaching of WO 2010/00447, the present invention provides a process for the production of the particles of the invention comprising:

a) forming of a solution of the two active ingredients in a pre-determined ratio in a suitable solvent;

b) generating an aerosol from the solution of said two active ingredients and drying the atomized droplets to yield unstable part-amorphous or fully amorphous particles;

c) collecting of the particles in a vessel containing an anti-solvent for both the active ingredients;

d) applying high intensity ultrasound to induce crystallisation of said two active ingredients into an individual particle; and

e) isolating and collecting the produced particles

As far as the step a) is concerned, the choice of the solvent is critical as, besides having a high solubilizing capacity for both the active ingredients, it should have a suitable degree of volatility and diffusion characteristics. These properties indeed significantly affect the (i) the particle size distribution of the particles; ii) condition of the particles for subsequent treatment as stated in step d); and (iii) the particle size distribution of the final isolated crystalline particles.

When the particles are prepared using the apparatus and techniques disclosed in WO 2004/073827, it has been found that a mixture of dichloromethane and methanol in a ratio comprised between 90:10 and 99:1 v/v is particularly suitable, as it gives rise to satisfactory results in terms of crystallinity degree, morphology and particle size distribution. Preferably mixtures of dichloromethane and methanol in ratios from 97:3 to 95:5 v/v are used. On the other hand, when the particles are prepared using the apparatus and techniques disclosed in WO 2010/00447, methanol turned out to be particularly suitable.

Varying the concentration of the active ingredients in the solution prepared in step a) of the process of the invention also affects the resultant particle size distribution. For the particles of the invention it has been found that is preferable to utilise an overall concentration of two active ingredients comprised between 0.8 and 5.0% w/v, preferably comprised between 1.0 and 3.0% w/v, more preferably between 1.5 and 2.0% w/v.

For step b), when the particles are prepared using the apparatus and techniques disclosed in WO 2004/073827, any aerosol based atomization system may be used for generation of the aerosol. Various systems for generating aerosols are well-known. The aerosol may, for example, be generated from the desired substance dissolved in a suitable solvent by electrohydrodynamic spraying, high air pressure atomizer, or other aerosol generators including pneumatic systems, rotary (spinning-top) systems, spray nozzles, nebulizers, propellant evaporation systems, piezoelectric transducers and ultrasonic transducers. For example, the aerosol may be generated using the electrohydrodynamic spraying system or the high-pressure atomization system. On the other hand, when the particles are prepared using the apparatus and techniques disclosed in WO 2010/00447, the solution is preferably atomized by spray-drying.

Controlling the conditions of the aerosol generation such as the temperature of the solution, the solution flow rate and the pressure of the carrier gas allows further control of the particle size distribution of the particles. Said conditions may be properly adjusted by the person skilled in the art in relation with the desired particle size distribution and the size of the batch.

In step c), the aerosol droplets are preferably collected in n-heptane as anti-solvent. When the particles are prepared using the apparatus and techniques disclosed in WO 2010/00447, other anti-solvents such as cyclohexane and 2-propanol and fluorinated hydrocarbons, such as perfluorodecalin, may also be used. In particular, perfluorodecalin is another preferred solvent.

The collection vessel is preferably a temperature-controlled collection vessel. Advantageously, when the particles are prepared using the apparatus and techniques disclosed in WO 2004/073827, the temperature of the anti-solvent is maintained below 10° C., preferably between 5 and 8° C., more preferably at about 5° C. On the other hand, when the particles are prepared using the apparatus and techniques disclosed in WO 2010/00447, the temperature of the anti-solvent may be maintained between 25° C. and 80° C., preferably between 55° C. and 75° C.

The volume of the anti-solvent is generally at least slightly larger than that of the solvent and their ratio is advantageously comprised between 1.5:1 and 10:1 v/v, preferably from 5:1 to 2:1.

In step d), ultrasonic energy is applied to the droplets upon collection of the droplets in the anti-solvent to induce nucleation and subsequent crystallisation of the droplets thus generating the crystalline particles of the invention.

The ultrasonic energy may be applied continuously or in a discontinuous manner such as by pulsed application. Any suitable source of ultrasonic vibration may be used. An ultrasonic probe may, for example, be inserted into the collection vessel, an ultrasonic emitter may be contained in the collection vessel or the collection vessel may be housed in an ultrasonic bath.

The amplitude and frequency of the ultrasound waves affects the rate of nucleation and crystal growth. The frequency of the ultrasound waves may for example be from 10 kHz to 1 MHz, preferably from 10 kHz to 500 kHz, more preferably from 10 kHz to 100 kHz such as at 10, at 20, 40, 60, 80, or 100 kHz or at any frequency therein between, such as, about 30 kHz or about 50 kHz.

The ultrasonic irradiation is employed at an amplitude that is appropriate for the formation of crystals of the desired size. For laboratory probe systems with an emitting face of, for example 8 cm², the amplitude selected may be from about 1 to 30 μm, typically from 3 to 20 μm, preferably from 5 to 10 μm. Probes having a probe face surface area of 80 cm² and a power requirement of from 5 to 80 W, provide a power density of from 0.6 to 12.5 W/cm² using an amplitude of 2 to 15 mm. In larger systems, comprising transducers bonded onto the flow cell, for example a 6 litre flow cell, the power density for the transducers employed may be from 10 to 100 W/L, preferably from 30 to 80 W/L, and more preferably from 50 to 75 W/L, for example about 60 W/L or about 70 W/L. In some embodiments of the invention, an ultrasonic probe operating at the frequency of 20 kHz and at a power of 20 to 40 W was advantageously used.

In step e), the particles obtained at the end of the crystallisation stage may be isolated from the resulting slurry and collected according to methods well known in the art. Advantageously the particles of the invention may be isolated by supercritical carbon dioxide extraction or by spray-drying, preferably by spray-drying. It has indeed been found that the particles obtained after isolation by spray-drying exhibit better flow properties.

In another aspect, the present invention provides a formulation for administration by inhalation comprising the particles of the invention. The particles may be formulated together with one or more pharmaceutically acceptable excipients, additives, diluents or carriers.

For example, the formulation may be provided in the form of suspension in a propellant as aerosol carrier to be administered by pressurized meted dose inhalers (pMDI). The pMDI comprises a canister wherein the formulation is filled and a metering valve for delivering a daily therapeutically effective dose of the formulation.

In certain embodiments the aerosol carrier may consist of a non-chlorofluorocarbon-based propellant such as hydrofluoralkane (HFA). In particular, the propellants HFA 134a and HFA 227, or mixtures thereof, may be advantageously used. The suspension formulation may comprise additional excipients such as surfactants, and wetting agents.

In a preferred embodiment, the formulation is provided in the form of dry powder for inhalation, more preferably in the form of an interactive or ordered mixture, by diluting the particles of the invention in a pharmacologically inert physiologically acceptable excipient consisting of coarser particles. Advantageously, said powder formulation for inhalation may comprise the particles according to the invention and coarse particles of a physiologically acceptable excipient, e.g. particles having a MMD higher than 90 microns and preferably the MD comprised between 50 microns and 500 microns, more preferably between 150 and 400 microns, even more preferably between 210 and 355 microns. In another embodiment, the coarse particles have a MD comprised between 90 and 150 microns.

In one of the preferred embodiments, when their MD is comprised between 210 and 355 micron, the coarse excipient particles have preferably a relatively highly fissured surface.

Preferably, the relevant powder formulation may further comprises a fraction of pharmacologically-inert microparticles having a MMD lower than 35 microns composed of particles of a physiologically acceptable excipient and an additive material selected from the class of the anti-adherents such as the amino acids leucine and isoleucine or of the lubricants such as magnesium stearate, sodium stearyl fumarate, stearyl alcohol, stearic acid, and sucrose monopalmitate.

More preferably, said powder formulation comprises a fraction of said pharmacologically-inert microparticles having a MMD lower than 15 microns, preferably lower than 10 microns, composed of particles of a physiologically acceptable excipient and particles of magnesium stearate according to the teaching of EP 1 274 406, which is incorporated herein by reference in its entirety.

In another preferred embodiment of the invention, when their MD is comprised between 90 and 150 microns, the coarse carrier particles have preferably a surface rugosity expressed as the fractal dimension of less than or equal to 1.1, determined according to the teaching of EP 1 196 146, which is incorporated herein by reference in its entirety. More preferably, the surface of said particles is coated with magnesium stearate.

Magnesium stearate is added to the formulations herein described with the aim of improving the respirable fraction of the active substance.

The physiologically acceptable excipient may be constituted of any amorphous or crystalline physiologically acceptable pharmacologically-inert material of animal or vegetal source or combination thereof. Preferred materials are crystalline sugars and for example monosaccharides such as glucose or arabinose, or disaccharides such as maltose, saccharose, dextrose or lactose. Polyalcohols such as mannitol, sorbitol, maltitol, lactitol may also be used. The most preferred material is α-lactose monohydrate. Examples of commercial lactose are Capsulac™ and Pharmatose™. An example of commercial mannitol is Pearlitol™.

In a preferred embodiment, the fraction of microparticles is composed of 98% by weight of α-lactose monohydrate and 2% by weight of magnesium stearate and the ratio between the fraction of microparticles and the fraction of coarse particles made of α-lactose monohydrate particles is 10:90% by weight, respectively.

The amount of magnesium stearate in the final formulation is advantageously comprised between 0.02% and 1.0% by weight, based on the total weight of the formulation, preferably between 0.05 and 0.5% by weight, more preferably between 0.1 and 0.4% by weight, even more preferably between 0.2 and 0.3% by weight.

The powder formulation for inhalation comprising the powder particles according to the present invention is characterized by a high degree of homogeneity. After mixing, the content uniformity of the active ingredient, expressed as relative standard deviation (RSD), is less than 5%, preferably equal to or less than 3.5%, more preferably equal to or less than 1.5%.

Said powder formulation may be administered by inhalation with any type of DPIs known in the art. DPIs can be divided into two basic types: i) single dose inhalers, for the administration of pre-subdivided single doses of the active compound; ii) multidose dry powder inhalers (MDPIs), either with pre-subdivided single doses or pre-loaded with quantities of active ingredient sufficient for multiple doses. On the basis of the required inspiratory flow rates (l/min) which in turn are strictly dependent on their design and mechanical features, DPIs are divided into: i) low-resistance devices (>90 l/min); ii) medium-resistance devices (about 60 l/min); and iii) high-resistance devices (about 30 l/min).

The particles of the present invention are indicated for the prevention and/or treatment of inflammatory or obstructive airways diseases such as asthma and chronic obstructive pulmonary disease (COPD). Other respiratory disorders characterized by obstruction of the pheripheral airways as a result of inflammation and/or presence of mucus such as chronic obstructive bronchiolitis, bronchiectasies, and cystic fibrosis may also benefit by their use.

Other features of the invention will become apparent in the course of the following descriptions of exemplary embodiments which are given for illustration of the invention and are not intended to be limiting thereof.

### Examples

### Example 1

### Preparation of Crystalline Particles of Carmoterol Hydrochloride and Budesonide in a Ratio 1:100 Using the Apparatus and Techniques Disclosed in WO 2004/073827

The high pressure atomization system described in the co-pending application, WO 2004/073827, is used to generate the aerosol. A 1.5% w/v solution is prepared by dissolving 8.19 g of carmoterol hydrochloride and budesonide in a ratio 1:100 w/w in a mixture of dichloromethane: methanol 95:4.5 v/v. Said solution, maintained at room temperature, is sprayed through a 0.7 mm diameter orifice with a supporting nitrogen flow rate of 11.5 ml/min. The flow rate of the solvent is controlled by a syringe pump and was set at 2 ml/min. The aerosol droplets are collected in 500 ml of n-heptane maintained at 5° C. via a conical shaped crystallisation vessel. The distance between the atomizer orifice and the collection vessel is pre-set at well-defined separation distances. A typical separation distance is around 15 cm. The whole system is hermetically sealed. Nucleation of the droplets collected in the crystallization vessel is induced via ultrasonic energy by inserting an ultrasonic probe operating at the frequency of 20 kHz and at a power of 40 W. The crystalline particles are isolated from the resulting slurry by supercritical carbon dioxide extraction, collected by filtration, washed with n-heptane, and subsequently dried. The yield is 1.53 g (18.6%).

### Example 2

### Characterisation of the Particles of Example 1

FIG. 1 shows a SEM image of the particles obtained in Example 1. It can be clearly observed that said particles are significantly distinct when compared to the SEM images of the two starting raw materials reported in FIG. 2, i.e. carmoterol hydrochloride and budesonide.

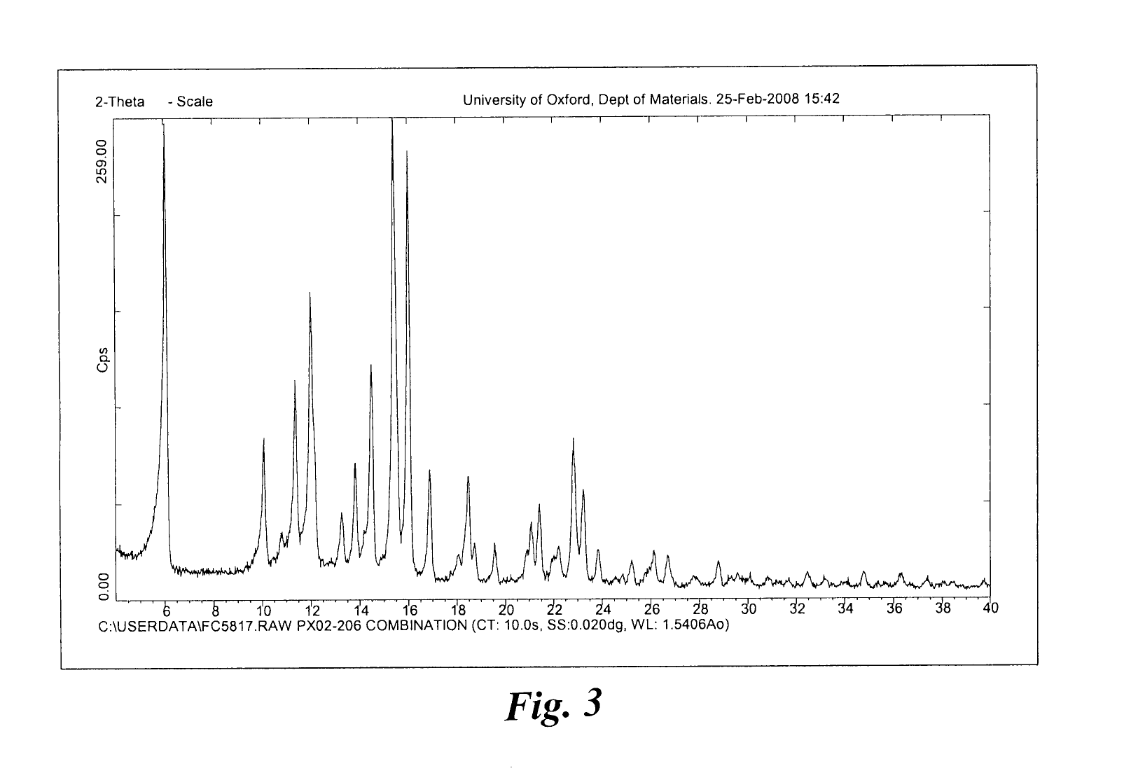

The obtained particles have also been characterised by X-ray powder diffractometry and differential scanning calorimetry (DSC). The X-ray powder diffraction (XPRD) pattern, reported in FIG. 3, shows characteristic sharp diffraction peaks associated with highly crystalline material consistent with budesonide. Unsurprisingly, no evidence for diffraction peaks corresponding to carmoterol hydrochloride could be observed due to its low amount.

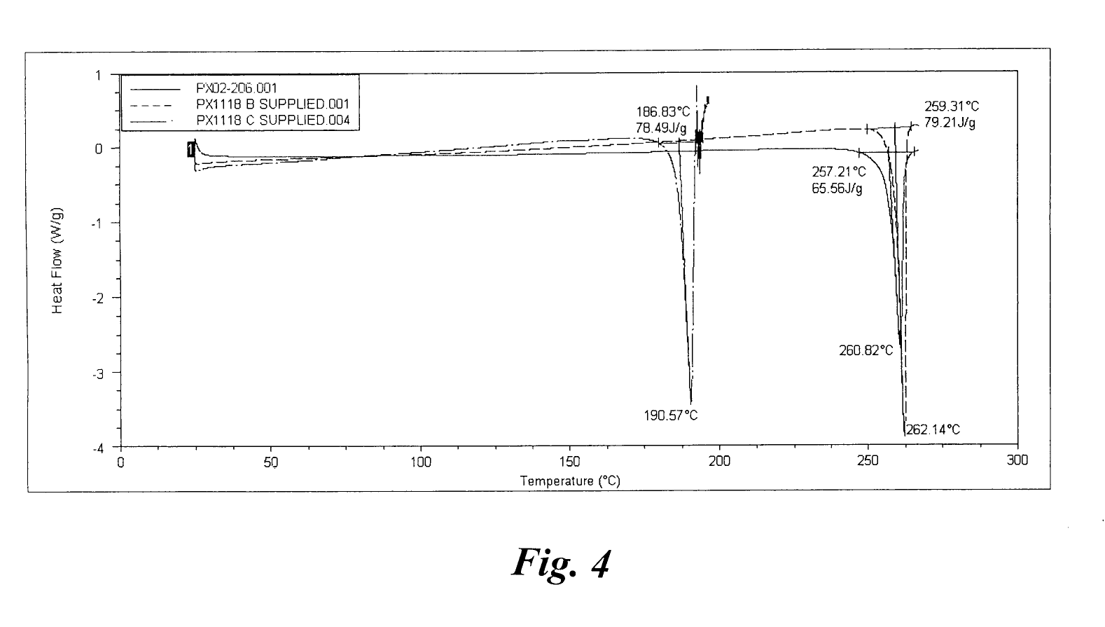

The thermogram, reported in FIG. 4, shows the characteristic endothermic transition at approximately 260° C., which corresponds to the melting point of budesonide. The melting point is indeed slightly lower than that of pure crystalline budesonide (about 262° C.). Said melting point depression is indicative of the incorporation of another component, wherein the low amount of carmoterol hydrochloride acts as an impurity.

The obtained particles have also been characterised in terms of particle size distribution. The particle size has been determined by laser diffraction using a Mastersize X apparatus. The parameters taken into consideration are the volume diameters (VD) in micron of 10%, 50% and 90% of the particles expressed as d(v,0.1), d(v,0.5), and d(v,0.9), respectively, which correspond to the mass diameter assuming a size independent density for the particles. The mean values of eight samples are reported in Table 1. The standard deviation (S.D.) turns out to be less than ±0.2.

TABLE 1
Particle size distribution.

| Particle size (μm) | Particles of Example 1 |
|---|---|
| d(v, 0.1) | 1.48 |
| d(v, 0.5) | 4.67 |
| d(v, 0.9) | 10.57 |

### Example 3

### Moisture Uptake Experiments

Dynamic vapour sorption (DVS) studies have been performed on the particles of Example 1 with a DVS-1 Instrument (Surface Measurement Systems Ltd, London UK). Approximately 50 mg of material is weighed into the sample pan of the DVS and exposed to one 0 to 90% relative humidity (RH) cycle (10% RH increments). The moisture sorption isotherms of the sample indicate a maximum water uptake at 90% RH of 0.35% w/w. The desorption isotherm shows that the sample has retained 0.07% w/w water which is indicative of a good stability profile on prolonged storage.

### Example 4

### “Interactive Ordered Mixture” Formulation Comprising the Particles of Example 1

The particles as obtained in Example 1 have been added to a carrier prepared according to the teaching of EP 1 274 406 and reported hereafter.

### a) Preparation of the Fraction of the Pharmacologically-Inert Microparticles.

α-lactose monohydrate SpheroLac™ 100 with a starting mass diameter of 50 to 400 microns (MMD of about 170 microns) and magnesium stearate particles in the ratio 98:2 percent by weight are co-milled in a jet mill apparatus until the MMD of the whole mixture is less than 15 microns.

### b) Addition of the Fraction of Microparticles to the Fraction of Coarse Particles.

90 percent by weight of α-lactose monohydrate CapsuLac™ (212 to 355 microns) is placed in a 240 ml stainless steel container, then 10 percent by weight of the fraction of pharmacologically-inert microparticles is added. The blend is mixed in a Turbula mixer for 2 hours at 42 r.p.m. to obtain the carrier.

### c) Addition of the Particles of Example 1 to the Carrier.

The particles are added to the carrier in a suitable amount in order to obtain a ratio of 1+100 μg of carmoterol hydrochloride +budesonide to 10 mg of final formulation. The resulting blend is mixed in a Turbula mixer for 30 min at 46 r.p.m.

### Example 5

### Characterisation of the Powder Formulation of Example 4

The powder formulation of Example 4 has been characterized in terms of the uniformity of distribution of the active ingredient and aerosol performances after loading it in the multidose dry powder inhaler Pulvinal™. The uniformity of distribution of the active ingredients is evaluated by withdrawing six samples from different parts of the blend and evaluated by HPLC. The evaluation of the aerosol performance is carried out using the Andersen Cascade Impactor (Apparatus D) according to the conditions reported in the European Pharmacopeia 6^{th} Ed 2008, par 2.9.18, pages 293-295.

After aerosolization of 10 doses, the ACI apparatus is disassembled and the amounts of drug deposited in the stages were recovered by washing with a solvent mixture and then quantified by High-Performance Liquid Chromatography (HPLC). The following parameters, are calculated: i) the delivered dose which is the amount of drug delivered from the device recovered in the impactor; ii) the fine particle dose (FPD) which is the amount of delivered dose recovered in the S3-AF stages having a particle size equal to or lower than 5.0 micron; iii) the fine particle fraction (FPF) which is the percentage of the fine particle dose; and iv) the MMAD. The results in terms of uniformity of distribution and aerosol performances (mean value±S.D) are reported in Table 2.

TABLE 2
Uniformity of distribution of the active ingredients
(.a.i.) and aerosol performances.

| Property | Value |
|---|---|
| Uniformity of distribution of the a.i. (μg) | |
| carmoterol hydrochloride | 1.25 ± 0.02 |
| budesonide | 101.12 ± 1.91 |
| Delivered dose (μg) | |
| carmoterol hydrochloride | 1.26 ± 0.01 |
| budesonide | 103.06 ± 0.42 |
| Fine particle dose (FPD, μg) | |
| carmoterol hydrochloride | 0.29 ± 0.05 |
| budesonide | 34.80 ± 2.86 |
| Fine particle fraction (FPF, %) | |
| carmoterol hydrochloride | 42.39 ± 1.00 |
| budesonide | 35.14 ± 1.55 |
| MMAD (μm) | |
| carmoterol hydrochloride | 2.77 ± 0.13 |
| budesonide | 3.66 ± 0.10 |

The formulation prepared using the particles of Example 1 shows an excellent uniformity of distribution of the active ingredients. In fact, the content uniformity of both active ingredients, expressed as relative standard deviation (RSD), is less than 2%.

From the delivered dose values, it can also be appreciated that the formulation provides a controlled delivery of the combination of carmoterol with budesonide, as the ratio of the drugs in each dose is substantially the same as that present in the particles before delivery. Finally, the formulation shows good aerosol performances in terms of respirable fraction with more than 30% of FPF for both the active ingredients.

### Example 6

### Preparation of Crystalline Particles of Carmoterol Hydrochloride and Budesonide in a Ratio 1:100 Using the Apparatus and Techniques Disclosed in WO 2010/007447 (PX1)

A 2.5% w/v solution was prepared by dissolving carmoterol hydrochloride and budesonide in a ratio 1:100 w/w in methanol. The solution was atomized and droplets subsequently dried using a Büchi laboratory-scale spray-drier. The typical process consisted of atomizing the methanol solution using: (i) the 100% aspirator setting (which equates to approximately 35 to 40 m³/hr gas flow-rate); (ii) the 30% pump setting (which equates to 9 to 10 mL/min) and (iii) an inlet temperature approximately 20° C. greater than the boiling point of selected system. An atomization pressure of 3 to 3.5 bar was used for the pressure with the gas flow rate of typically 10 L/min. The generated unstable particles were collected in an ultrasonic chamber filled with n-heptane maintained at 25° C. Nucleation of the unstable particles collected in the ultrasonic chamber was induced via ultrasonic energy by using ultrasonic probe operating at the frequency of 20 kHz and at a power of 20 W. The final crystalline particles were isolated by supercritical carbon dioxide extraction, collected by filtration, washed with n-heptane, and subsequently dried. The yield is 0.7 g.

### Example 7

### Preparation of Crystalline Particles of Carmoterol Hydrochloride and Budesonide in a Ratio 1:100 Using the Apparatus and Techniques Disclosed in WO 2010/007447 (PX2)

A 2.5% w/v solution is prepared by dissolving carmoterol hydrochloride and budesonide in a ratio 1:100 w/w in methanol. The solution was atomized using a Büchi laboratory-scale spray-drier. The typical process consisted of atomizing the methanol solution using: (i) the 100% aspirator setting (which equates to approximately 35 to 40 m³/hr gas flow-rate); (ii) the 30% pump setting (which equates to 9 to 10 mL/min) and (iii) an inlet temperature approximately 20° C. greater than the boiling point of selected system. An atomization pressure of 3 to 3.5 bar was used for the pressure with the gas flow rate of typically 10 L/min. The generated unstable particles were collected in an ultrasonic chamber filled with n-heptane maintained at 55° C. Nucleation of the unstable particles collected in the ultrasonic chamber was induced via ultrasonic energy by using ultrasonic probe operating at the frequency of 20 kHz and at a power of 20 W. The crystalline particles are isolated by spray-drying and subsequently dried. The yield is 1.0 g.

### Example 8

### Preparation of Crystalline Particles of Carmoterol Hydrochloride and Budesonide in a Ratio 1:100 According to the Teaching of WO 2010/007447 (PX3)

The particles were prepared as described in Example 7 but using perfluorodecalin as anti-solvent maintained at 75° C. The crystalline particles were isolated by filtration. The yield is 1.0 g.

### Example 9

### Preparation of Crystalline Particles of Carmoterol Hydrochloride and Budesonide in a Ratio 1:50 Using the Apparatus and Techniques Disclosed in WO 2010/007447 (PX4)

The particles are prepared as described in Example 7 but by using a ratio of carmoterol hydrochloride and budesonide of 1:50 w/w. The yield is 1.1 g.

### Example 10

### Characterisation of the Particles of Examples 6, 7, 8, and 9

The content of the two active ingredients in each sample is determined by HPLC. The results are reported in Table 3.

TABLE 3
Content % (±S.D.) of the two active ingredients in the samples.

| Sample | % of both a.i. | Carmoterol HCl (%) | Budesonide (%) |
|---|---|---|---|
| PX 1 | 99.33 | 1.04 ± 0.01 | 98.29 ± 0.35 |
| PX 2 | 97.61 | 0.90 ± 0.09 | 96.71 ± 0.18 |
| PX3 | 97.43 | 0.94 ± 0.02 | 96.50 ± 0.42 |
| PX 4 | 97.60 | 1.94 ± 0.06 | 95.66 ± 0.57 |

The samples have also been characterised by differential scanning calorimetry (DSC) as reported in Example 2. The thermogram of all the particles shows a melting point slightly lower than that of pure crystalline budesonide which is indicative of the incorporation of carmoterol hydrochloride. It can be appreciated that the content of the active ingredients corresponds to the expected ratio, i.e. 1:100 for PX1, PX2, PX3, and 50:1 for PX4.

The particle size distribution has been determined as reported in Example 2. The values are reported in Table 4.

TABLE 4
Particle size distribution.

| Sample | dv 0.1 (μm) | dv 0.5 (μm) | dv 0.9 (μm) |
|---|---|---|---|
| PX 1 | 0.83 | 1.77 | 3.07 |
| PX 2 | 0.85 | 2.03 | 2.78 |
| PX3 | 0.85 | 1.42 | 1.93 |
| PX 4 | 1.91 | 3.37 | 6.13 |

### Example 11

### Characterization of Powder Formulations Comprising the Particles of Examples 6, 7, 8, and 9

The powder formulations have been prepared as reported in Example 4 and characterized in terms of the uniformity of distribution of the active ingredient and aerosol performances as reported in Example 5. The results are reported in Table 5.

TABLE 5
Uniformity of distribution of the active ingredients (a.i.) and aerosol performances.

| Property | PX1 | PX2 | PX3 | PX4 |
|---|---|---|---|---|
| Uniform. of distribution of the | | | | |
| a.i. (μg) | | | | |
| carmoterol hydrochloride | 1.13 ± 0.03 | 1.04 ± 0.03 | 1.03 ± 0.07 | 2.04 ± 0.07 |
| Budesonide | 101.7 ± 2.0 | 102.4 ± 1.3 | 104.3 ± 2.3 | 100.8 ± 2.9 |
| Delivered dose (μg) | | | | |
| carmoterol hydrochloride | 1.22 ± 0.03 | 1.01 ± 0.06 | 1.08 ± 0.09 | 2.07 ± 0.25 |
| Budesonide | 109.8 ± 3.0 | 108.2 ± 7.8 | 111.98 ± 6.2 | 102.3 ± 12.2 |
| Fine particle dose (FPD, μg) | | | | |
| carmoterol hydrochloride | 0.87 ± 0.03 | 0.48 ± 0.01 | 0.82 ± 0.05 | 0.74 ± 0.11 |
| Budesonide | 78.96 ± 4.75 | 44.62 ± 0.63 | 79.27 ± 1.2 | 28.17 ± 2.59 |
| Fine particle fraction (FPF, %) | | | | |
| carmoterol hydrochloride | 78.93 ± 2.04 | 49.98 ± 4.02 | 85.53 ± 1.5 | 43.27 ± 0.48 |
| Budesonide | 77.18 ± 1.40 | 44.62 ± 0.63 | 82.48 ± 5.3 | 36.53 ± 1.05 |
| MMAD (μm) | | | | |
| carmoterol hydrochloride | 1.65 ± 0.02 | 2.63 ± 0.29 | 1.38 ± 0.04 | 2.92 ± 0.02 |
| Budesonide | 1.61 ± 0.05 | 2.88 ± 0.08 | 1.35 ± 0.13 | 2.99 ± 0.16 |

The formulations prepared using the particles of Examples 6, 7, 8, and 9 show an excellent uniformity of distribution of the active ingredients. In fact, the content uniformity of carmoterol hydrochloride, expressed as relative standard deviation (RSD), is less than 1%, while that of budesonide is less than 3%.

From the delivered dose values, it can also be appreciated that all formulation provide a controlled delivery of the combination of carmoterol with budesonide, as the ratio of the drugs in each dose is substantially the same as that present in the particles before delivery. Finally, all formulations show good aerosol performances in terms of respirable fraction with more than 30% of FPF for both the active ingredients.

Finally, after storage under long-term conditions (25° C., 60% r.h.) for three months, both active ingredients in the formulations turned out to be chemically stable and no significant change in the respirable fraction was observed

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. Crystalline particles, which comprise a combination of a pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone and a corticosteroid in a ratio of no more than 1:50.

2. Crystalline particles according to claim 1, wherein said corticosteroid is selected from the group consisting of beclometasone dipropionate, budesonide, ciclesonide, mometasone, furoate ester of mometasone, fluticasone, propionate ester of fluticasone, and furoate ester of fluticasone.

3. Crystalline particles according to claim 1, wherein said ratio is from 1:50 to 1:800.

4. Crystalline particles according to claim 3, wherein said ratio is from 1:80 to 1:400.

5. Crystalline particles according to claim 4, wherein said corticosteroid is budesonide.

6. A process for preparing crystalline particles according to claim 1, comprising:
(a) forming a solution of said pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone and said corticosteroid in a pre-determined ratio in a solvent;
(b) generating an aerosol from said solution;
(c) collecting droplets of said aerosol in a vessel containing an anti-solvent for said pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone and said corticosteroid;
(d) applying high intensity ultrasound to induce crystallisation of said pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone and said corticosteroid into individual particles; and
(e) isolating and said particles.

7. A process for preparing crystalline particles according to claim 1, comprising:
(a) forming a solution of said pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone and said corticosteroid in a pre-determined ratio in a solvent;
(b) generating an aerosol from the solution of said two active ingredients and drying the atomized droplets to yield unstable part-amorphous or fully amorphous particles;
(c) collecting said particles in a vessel containing an anti-solvent for said pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone and said corticosteroid;
(d) applying high intensity ultrasound to induce crystallisation of said pharmaceutically acceptable salt of 8-hydroxy-5-[(1R)-1-hydroxy-2-[[(1R)-2-(4-methoxyphenyl)-1-methylethyl]amino]ethyl]-2(1H)-quinolinone and said corticosteroid into individual particles; and
(e) isolating and said particles.

8. A process according to claim 6, wherein said particles are isolated by spray-drying.

9. A process according to claim 7, wherein said particles are isolated by spray-drying.

10. A pharmaceutical formulation, comprising crystalline particles according to claim 1 and one or more pharmaceutically acceptable excipients.

11. A pharmaceutical formulation, comprising crystalline particles according to claim 2 and one or more pharmaceutically acceptable excipients.

12. A pharmaceutical formulation, comprising crystalline particles according to claim 3 and one or more pharmaceutically acceptable excipients.

13. A pharmaceutical formulation, comprising crystalline particles according to claim 4 and one or more pharmaceutically acceptable excipients.

14. A pharmaceutical formulation, comprising crystalline particles according to claim 5 and one or more pharmaceutically acceptable excipients.

15. A pharmaceutical formulation according to claim 10, which is in the form of dry powder.

16. A dry powder inhaler, which contains a formulation according to claim 15.

17. A pharmaceutical formulation according to claim 10, which is in the form of a suspension of particles in a propellant.

18. A pressurized metered dose inhaler, comprising a canister which contains a formulation according to claim 17 and a metering valve for delivering a daily therapeutically effective dose of the formulation.

19. A method for the prevention and/or treatment of an inflammatory or obstructive airways disease, comprising administering to a subject in need thereof an effective amount of crystalline particles according to claim 1.

20. A method according to claim 19, wherein said disease is asthma or chronic obstructive pulmonary disease.

21. A method for the prevention and/or treatment of an inflammatory or obstructive airways disease, comprising administering to a subject in need thereof an effective amount of crystalline particles according to claim 2.

22. A method according to claim 21, wherein said disease is asthma or chronic obstructive pulmonary disease.
